(19) United States
(12) Patent Application Publication
(10) Pub. No.: US 2026/0135000 A1
(43) Pub. Date: May 14, 2026

(54) DETECTING HAND SANITATION USE WITH AUDIO SENSOR

(71) Applicant: Patient Voice AI, Inc., Prosper, TX (US)

(72) Inventors: Jordi Torras, Allen, TX (US); Arnau Torras, Allen, TX (US); Tyler Foust, Melissa, TX (US)

(21) Appl. No.: 19/085,017

(22) Filed: Mar. 20, 2025

Related U.S. Application Data
(60) Provisional application No. 63/720,093, filed on Nov. 13, 2024.

(51) Int. Cl. G16H 50/30; G16H 40/20; G16H 40/60; G16H 70/20

(52) U.S. Cl. CPC G16H 50/30; G16H 40/20; G16H 40/60; G16H 70/20

(57) ABSTRACT

A method for monitoring sanitation events in a medical environment includes receiving, at a monitoring device, a first signal indicative of the presence of a user device and receiving, at the monitoring device, a second signal generated by an audio sensor in communication with monitoring device. The second signal is indicative of a sanitation action by a user within audio range of the monitoring device. The method further includes determining, at the monitoring device, an event type based on the second signal, using both information from the first signal and the event type from the second signal as inputs to a model and calculating, based on an output of the model, an infection risk score.

### CROSS-REFERENCE TO RELATED APPLICATION(S)

This application claims priority to U.S. Provisional Application No. 63/720,093 filed on Nov. 13, 2024, the benefit of which is claimed and the disclosure of which is incorporated herein in its entirety. This application is related to U.S. Non-Provisional Application No. ______, titled “Detecting Hand Sanitation Use with Device Sensor”, the disclosure of which is incorporated herein in its entirety.

### FIELD OF THE DISCLOSURE

The present disclosure relates, in general, to sanitation monitoring, and more particularly, to devices, systems, and methods for monitoring hand sanitation in a medical facility.

### BACKGROUND

Maintaining high levels of sanitation in a medical facility is desirable for preventing or reducing the incidence of healthcare related infections with respect to patients in the medical facility and for remaining in compliance with various health and safety laws, codes, and regulations. One way in which sanitation is promoted in a medical facility is through frequent hand washing and frequent use of hand sanitation devices. Monitoring compliance with sanitation requirements, including hand sanitation, however, can be challenging.

### SUMMARY

Embodiments of the present disclosure include systems, devices, and methods of detecting and monitoring hand sanitation.

In one general aspect, the present disclosure is directed to a method for monitoring sanitation events in a medical environment. The method also includes receiving, at a monitoring device, a first signal indicative of the presence of a user device; receiving, at the monitoring device, a second signal generated by an audio sensor in communication with monitoring device, where the second signal is indicative of a sanitation action by a user within audio range of the monitoring device; determining, at the monitoring device, an event type based on the second signal; using both information from the first signal and the event type from the second signal as inputs to a model; and calculating, based on an output of the model, an infection risk score.

In some aspects, implementations may include one or more of the following features. The method where the first signal is a Bluetooth signal from the user device. The model is a machine-learning model that is produced by processing historical data that correlates a number of visitors and sanitation events with healthcare infections. The medical environment is an emergency room, an operating room, or a patient room of a hospital. The event type is determined by generating, using a neural network-based model, one or more classifications based on the second signal, where the one or more classifications indicate the source of a sound in the medical environment. The source is a sanitation device. The sanitation device is one of a hand sanitizer or a handwashing sink. The event type is determined by comparing the second signal with one or more audio templates. The infection risk score indicates a likelihood of a healthcare infection within the medical environment.

In one general aspect, the present disclosure is directed to a system. The system includes a memory configured to store instructions for a model. The system also includes a processor coupled to the memory and configured to read the instructions from the memory to cause the system to perform operations, the operations that may include: receive, at a monitoring device, a first signal indicative of the presence of a user device; receive, at the monitoring device, a second signal generated by an audio sensor in communication with monitoring device, where the second signal is indicative of a sanitation action by a user within audio range of the monitoring device; determine, at the monitoring device, an event type based on the second signal; use both information from the first signal and the event type from the second signal as inputs to the model; and calculate, based on an output of the model, an infection risk score.

In some aspects, implementations may include one or more of the following features. The system where the first signal is a Bluetooth signal from the user device. The model is a machine-learning model that is produced by processing historical data that correlates a number of visitors and sanitation events with healthcare infections. The medical environment is an emergency room, an operating room, or a patient room of a hospital. The operations may include determining the event type by generating, using a neural network-based model, one or more classifications based on the second signal, where the one or more classifications indicate the source of a sound in the medical environment. The source is a sanitation device. The sanitation device is one of: a hand sanitizer or a handwashing sink. The event type is determined by operations that may include comparing the second signal with one or more audio templates. The infection risk score indicates a likelihood of a healthcare infection within the medical environment.

In one general aspect, the present disclosure is directed to a non-transitory machine-readable medium that may include a plurality of instructions, executable by one or more processors, where the plurality of instructions are configurable to cause the one or more processors to perform operations comprising: receive, at a monitoring device, a first signal indicative of the presence of a user device; receive, at the monitoring device, a second signal generated by an audio sensor in communication with monitoring device, where the second signal is indicative of a sanitation action by a user within audio range of the monitoring device; determine, at the monitoring device, an event type based on the second signal; use both information from the first signal and the event type from the second signal as inputs to a model; and calculate, based on an output of the model, an infection risk score.

In some aspects, implementations may include one or more of the following features. The non-transitory machine-readable medium where the first signal is a Bluetooth signal from the user device.

Additional aspects, features, and advantages of the present disclosure will become apparent from the following detailed description.

### BRIEF DESCRIPTION OF THE DRAWINGS

FIG. 1 illustrates a sanitation monitoring system, according to one or more embodiments;

FIG. 2A illustrates a sanitation device in communication with a user device, according to one or more embodiments;

FIG. 2B illustrates an exemplary medical environment, according to one or more embodiments;

FIG. 3 illustrates a neural network-based model configuration for audio classification, according to one or more embodiments;

FIG. 4 illustrates exemplary audio templates, according to one or more embodiments;

FIG. 5 illustrates a display of sanitation compliance scores generated by the sanitation monitoring system of FIG. 1 for a user, according to one or more embodiments;

FIG. 6 illustrates a display of sanitation compliance scores generated by the sanitation monitoring system of FIG. 1 for a supervisor, according to one or more embodiments;

FIG. 7 illustrates a method of monitoring use of a sanitation device, according to one or more embodiments;

FIG. 8 illustrates a method of tracking hand sanitation of staff members in a medical facility using the sanitation monitoring system of FIG. 1, according to one or more embodiments;

FIG. 9 illustrates a method of calculating a probability of a healthcare related infection occurring with respect to a patient of the medical facility using the sanitation monitoring system of FIG. 1, according to one or more embodiments; and

FIG. 10 illustrates a node for implementing one or more embodiments of the present disclosure.

### DETAILED DESCRIPTION

The following disclosure provides many different embodiments. Specific examples of components and arrangements are described below to simplify the present disclosure. These are, of course, merely examples and are not intended to be limiting. In addition, the present disclosure may repeat reference terms in the various embodiments. This repetition is for the purpose of simplicity and clarity and does not in itself dictate a relationship between the various embodiments and/or configurations discussed.

In order to provide better hand sanitation monitoring, and healthcare infection risk assessment, the examples described herein compare detected hand sanitation usage and detected visitor data with models to predict infection risk. Hand sanitation may be detected, for example, through a sensor on a hand sanitation device such as a soap dispenser. Hand sanitation may also be monitored using an audio sensor. Visitor data may be monitored by detecting Bluetooth signals that enter and exit a room. The sanitation data and the visitor data may then be applied to a model that predicts healthcare infection risk. The model may be, for example, a machine learning model.

With the techniques described herein, a sanitation monitoring system may predict the risk of a healthcare infection. For example, if several persons enter a hospital room, and the hand sanitation usage is low, the system may predict a higher risk of infection. If, for example, the hand sanitation usage is higher, the system may predict a lower risk of infection. If several persons are in the room at the same time, the system may predict a higher risk of infection, according to the model.

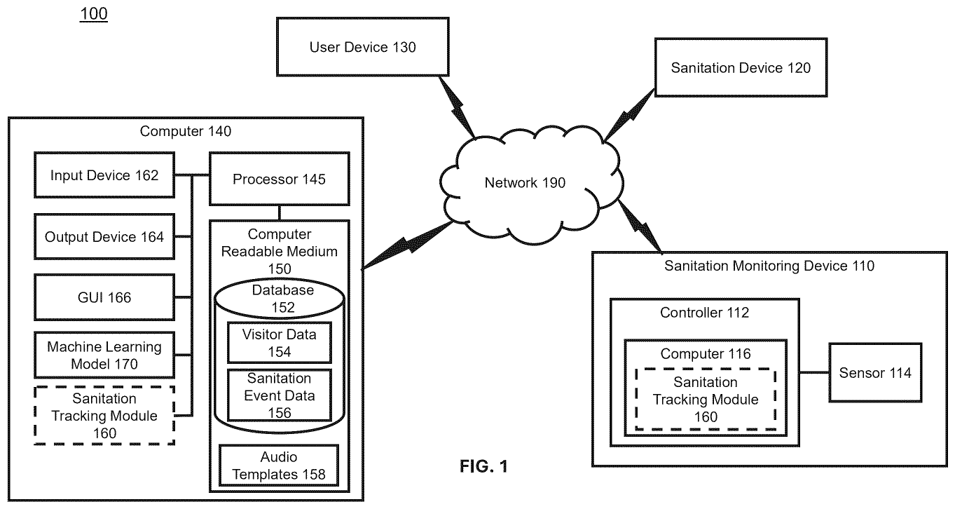

FIG. 1 illustrates a sanitation monitoring system 100, according to one or more embodiments of the present disclosure. In one or more embodiments, the sanitation monitoring system 100 may be associated with a medical facility, such as, for example, a hospital. The sanitation monitoring system includes a sanitation monitoring device 110, a sanitation device 120, a user device 130, and a computer 140. In one or more embodiments, each of the sanitation monitoring device 110, the sanitation device 120, the user device 130, and the computer 140 may be in communication with each other via a network 190. In one or more embodiments, one or more components of the sanitation monitoring system 100 may be positioned in various medical environments, including in a room of the medical facility, such as, for example, an emergency room, an operating room, a patient room, or a waiting room.

In one or more embodiments, the network 190 may include the Internet, one or more local area networks, one or more wide area networks, one or more cellular networks, one or more wireless networks, one or more voice networks, one or more data networks, one or more communication systems, and/or any combination thereof. In some embodiments, the network may also include Wi-Fi®, Bluetooth®, and Long-Term Evolution (“LTE”) or other wireless broadband communication technology.

In one or more embodiments, the sanitation device 120 may include a hand sanitation device, such as a manual or automatic hand sanitizer dispenser, a manual or automatic soap dispenser, a hand washing station, a sink, or other similar devices. In one or more embodiments, the sanitation device 120 may be equipped with a sensor or a communication device that is configured to send a signal to, or otherwise communicate to, the sanitation monitoring device 110 that the sanitation device 120 has been operated or used.

In one or more embodiments, the user device 130 may be, or include, a telephone, a tablet, a mobile device, a personal computer, a personal digital assistant, a cellular telephone or mobile phone, other types of telecommunications devices, other types of computing devices, and/or any combination thereof.

In one or more embodiments, the computer 140 includes a computer processor 145 and a computer readable medium 150 operably coupled thereto. Instructions accessible to, and executable by, the computer processor are stored on the computer readable medium. A database 152 is also stored in the computer readable medium 150. In one or more embodiments, the database 152 may store data, such as visitor data 154 and sanitation event data 156. In some aspects, one or more audio templates 158 may be stored in computer readable medium 150. In general, the computer 140 also includes an input device 162, an output device 164, and a graphical user interface (“GUI”) 166. In one or more embodiments, the computer may also store an application or module, such as a sanitation tracking module 160, which may be executed by the computer using the computer processor. In one or more embodiments, the computer 140 may be positioned remotely relative to the sanitation monitoring device 110. In one or more other embodiments, the computer may be positioned locally relative to the sanitation monitoring device.

The sanitation monitoring device 110 includes a controller 112 and a sensor 114. In one or more embodiments, the controller 110 may include a computer 116, such as, for example, the computer 140 described above. In one or more embodiments, the sensor 114 is an audio or sound sensor. In such case, the controller 112 may process the audio signals received from the audio sensor 114. Those received audio signals may then be compared with known audio profiles of hand sanitation usage. For example, the known audio profiles may represent the sound of the sink running, the sound of a hand sanitizer dispenser, the sound of a soap dispenser, etc.

In some embodiments, the sensor 114 may be a receiver that receives signals transmitted from a detector on a sanitation device 120. For example, a detector may be installed on a hand sanitizer dispenser or soap dispenser. When the dispenser is used, the detector may detect that event and transmit a signal to the sensor 114 on the sanitation monitoring device 110.

In one or more embodiments, the sanitation monitoring system 100 may be deployed in a patient room in a hospital. In one or more embodiments, the sanitation monitoring system 100 may be configured to track or monitor the number of visitors that enter the patient room. In one or more embodiments, the sanitation monitoring system 100 may be configured to monitor or track the number of hand sanitation events that occur in the patient room. In one or more embodiments, the sanitation monitoring system 100 may be configured to calculate, based on the tracking or monitoring of the number of visitors that enter the patient room and the number of hand sanitation events that occur in the patient room, the probability of a patient in the patient room experiencing a healthcare related infection. In one or more embodiments, visitors to the patient room may include medical staff affiliated with the hospital or guests unaffiliated with the hospital.

In one or more embodiments, the sanitation monitoring device 110 and the sanitation device 120 are positioned in a patient room. The sanitation monitoring device 110 is configured to detect when a visitor enters and exits the patient room. In one or more embodiments, the sanitation monitoring device 110 detects when a visitor enters and exits the patient room by detecting the presence of a user device 130 associated with the visitor. In one or more embodiments, the sanitation monitoring device 110 detects the user device 130 associated with the visitor by establishing a short-range wireless connection (e.g., a Bluetooth® connection) with the user device 130. In one or more embodiments, the sanitation monitoring device 110 may determine that the visitor entered the patient room (e.g., an entrance event) when the wireless connection with the user device 130 is established and may determine that the visitor exited the patient room (e.g., an exit event) when the wireless connection with the user device 130 is terminated, which may occur when the user device 130 is no longer in communication range with the sanitation monitoring device 110 because the user, along with the user device 130, has left the patient room. In one or more embodiments, the sanitation monitoring device 110 may detect a plurality of user devices associated with a plurality of users.

In one or more embodiments, the user device 130 is able to transmit to the sanitation monitoring device 110, and the sanitation monitoring device 110 is able to receive from the user device 130, information when the sanitation monitoring device 110 and the user device 130 are connected via the wireless connection. In one or more embodiments, the information may include visitor data. In one or more embodiments, the information may include identifying information with respect to the user associated with the user device 130. For example, in one or more embodiments, the information may identify the user associated with the user device 130 as a medical staff member affiliated with the hospital or as a guest unaffiliated with the hospital.

In one or more embodiments, the sanitation monitoring device 110 may include an internal counter, such as a visitor counter. In one or more embodiments, the sanitation monitoring device 110 may increment the internal counter by one each time the sanitation monitoring device 110 detects that a user or user device 130 has entered the patient room. In one or more embodiments, the sanitation monitoring device 110 may include two separate visitor counters, including a staff member counter for tracking the number of staff members that enter the patient room and a guest counter for tracking the number of guests that enter the patient room, which visitor counters may be added together to track the total number of visitors that enter the patient room. The counters facilitate the tracking or monitoring of the number of visitors that enter the patient room, visitors who may carry germs that put the patient in the patient room at risk of contracting a healthcare related infection.

In one or more embodiments, the sanitation monitoring device 110 may also track or monitor the number of uses of the sanitation device 120 in the patient room. In one or more embodiments, the sanitation monitoring device 110 may track or monitor the number of uses of the sanitation device 120 in the patient room by a specific visitor that has entered the patient room.

For example, in one or more embodiments, the sanitation monitoring device 110 detects that a visitor has entered the patient room when a wireless connection is established between the sanitation monitoring device 110 and the user device 130 associated with the visitor. While the wireless connection is established with the user device 130, the sanitation monitoring device 110 detects or receives a signal or information indicating that the sanitation device 120 has been used.

In one or more embodiments, the sanitation monitoring device 110 may be in communication with the sanitation device 120 via the network 190. In such embodiments, upon use or activation of the sanitation device 120 by the visitor, the sanitation device 120 sends a signal or otherwise communicates to the sanitation monitoring device 110 that the sanitation device 120 has been used or activated.

In one or more other embodiments, the sanitation monitoring device 110 may be equipped with an audio sensor 114 that is configured to detect the use or operation of the sanitation device by detecting sound waves associated with the use or operation of the sanitation device 120. In such embodiments, the sanitation monitoring device 110 or the sensor 114 may be trained to recognize an audio profile associated with the use, activation, or operation of the sanitation device 110. For example, in one or more embodiments, a hand sanitizer dispenser or a sink may have a unique and recognizable audio profile that can be learned by the sanitation monitoring device such that the sanitation monitoring device is capable of detecting the use of such devices. In one or more embodiments, the sanitation monitoring device may be trained using a machine learning model 170. In one or more embodiments, the machine learning model 170 may classify received audio samples, e.g., in the form of a spectrogram, for classification to determine the type of event the sound represents. For example, many sounds detected within a medical environment may not be relevant to sanitation, e.g., patient talking, medical equipment sounds, etc. However, some sounds, such as those made by sanitation devices are relevant to determining if sanitations procedures have been followed, e.g., hand washing compliance.

While the wireless connection is established between the sanitation monitoring device 110 and the user device 130 associated with the visitor that has entered the patient room, the sanitation device 120 may be used or operated, which operation may be detected by the sanitation monitoring device 110 in the ways described above. Upon detecting the use or operation of the sanitation device 120, the sanitation monitoring device 110 may attribute or associate such a use or operation with the user device 130 that is in wireless communication with the sanitation monitoring device 110 when the use or operation of the sanitation device 120 is detected, and thus also attribute or associate such a use or operation with the visitor associated with the user device 130 and in the patient room. Thus, the sanitation monitoring device 110 is able to track or monitor whether the visitor in the patient room has used the sanitation device 120 to wash or otherwise sanitize their hands.

In one or more embodiments, the sanitation monitoring device 110 may be configured to track or monitor whether a visitor uses the sanitation device 120 upon entering the patient room and upon exiting the patient room. For example, in one or more embodiments, the sanitation monitoring device 110 may detect the use of the sanitation device 120 shortly after or proximate to the time when the wireless communication between the sanitation monitoring device 110 and the user device 130 is established. The sanitation monitoring device 110 may recognize this occurrence and associate such a use of the sanitation device 120 with the visitor entering the patient room. For further example, in one or more embodiments, the sanitation monitoring device 110 may detect the use of the sanitation device 120 shortly before or proximate to the time when the wireless communication between the sanitation monitoring device 110 and the user device 130 is terminated. The sanitation monitoring device 110 may recognize this occurrence and associate such a use of the sanitation device 120 with the visitor exiting the patient room. Thus, the sanitation monitoring device 110 is able to track or monitor whether the visitor in the patient room used the sanitation device 120 to sanitize their hands upon entering the patient room and upon exiting the patient room.

In one or more embodiments, the sanitation monitoring device 110 may include another internal counter, such as a sanitation event counter. In one or more embodiments, the sanitation monitoring device 110 may increment the internal counter by one each time the sanitation monitoring device 110 detects that the sanitation device 120 has been used. In one or more embodiments, the sanitation monitoring device 110 may include two separate sanitation event counters, including an entrance sanitation event counter for tracking the number of times the sanitation device 120 is used when a visitor enters the patient room (a pre-visit sanitation event) and an exit sanitation event counter for tracking the number of times the sanitation device 120 is used when a visitor exits the patient room (a post-visit sanitation event), which sanitation event counters may be added together to track the total number of uses of the sanitation device 120 in the patient room. The counters facilitate the tracking or monitoring of the number of uses of the sanitation device 120 in the patient room, which may be compared to the number of visitors that enter the patient room in order to analyze or evaluation a sanitation level of the patient room and to analyze or evaluate the risk level associated with the patient in the patient room experiencing a healthcare related infection.

In one or more embodiments, any of the information described above, including visitor count information, visitor identity information, sanitation device use count information, specific visitor use of the sanitation device information, etc., may be stored as data, such as visitor data and sanitation event data, in the database 152 of the computer, which may be positioned locally or remotely relative to the sanitation monitoring device 110. In one or more embodiments, this information may also be transmitted to or stored on the user devices of visitors who are medical staff affiliated with the medical facility in which the sanitation monitoring system 100 is deployed. In one or more embodiments, this information may also be displayed on the computer 140, the sanitation monitoring device 110, or the user device 130.

The visitor data and the sanitation event data collected by the sanitation monitoring device 110 as described above may be used to calculate a sanitation compliance score. In one or more embodiments, the sanitation compliance score may be associated with the patient room generally and may indicate the frequency with which visitors to the patient room use the sanitation device 120, the percentage of visitors that use the sanitation device 120 in the patient room, the percentage of visitors that use the sanitation device 120 when entering the patient room, or the percentage of visitors that use the sanitation device 120 when exiting the patient room.

In one or more embodiments, a sanitation compliance score may be calculated for each individual visitor that enters the patient room. For example, in one or more embodiments, a sanitation compliance score may be calculated for each individual staff member of the hospital and may indicate, as a percentage, the number of times the staff member uses the sanitation device compared to the number of times the staff member enters a patient room. In one or more embodiments, the sanitation compliance score may include a score for the number of times the staff member uses the sanitation device when entering a patient room, a score for the number of times the staff member uses the sanitation device when exiting a patient room, and a total or overall score indicating overall compliance. In one or more embodiments, the score may be relative to a compliance requirement. For example, in one or more embodiments, the compliance requirement may be that a staff member must use a sanitation device at least once upon entering a patient room or that a staff member must use a sanitation device at least once upon entering a patient room and at least once upon exiting a patient room.

In one or more embodiments, any of the information collected or any of the sanitation compliance scores calculated may be transmitted to or displayed via the sanitation monitoring device 110, the computer 140, or the user device 130.

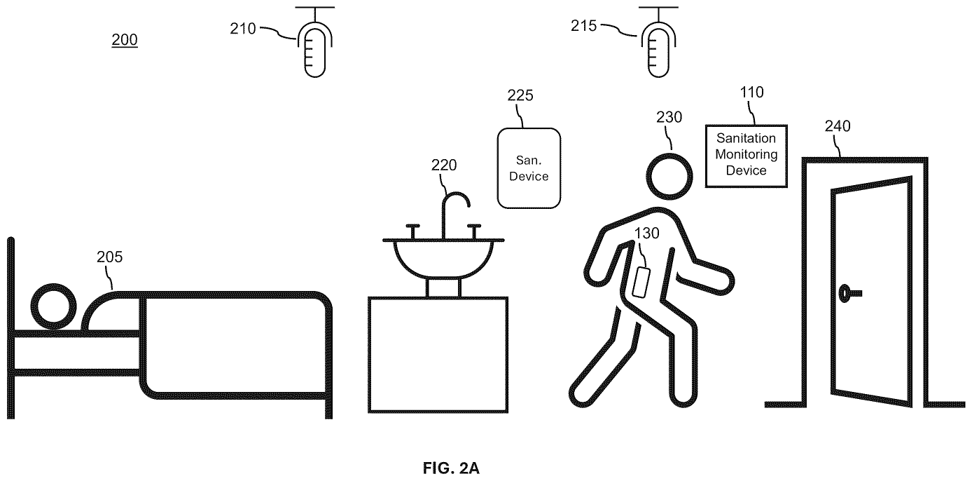

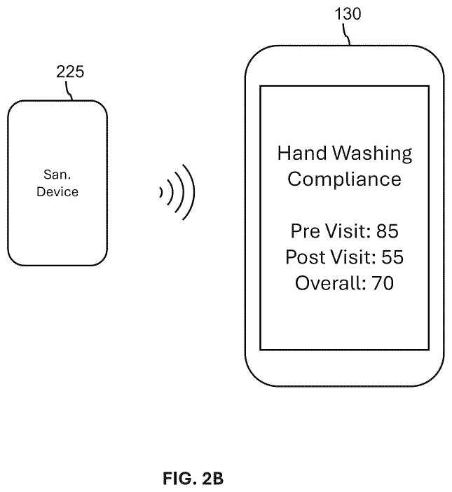

FIG. 2A illustrates an exemplary medical environment 200. One or more components of sanitation monitoring system 100, described in FIG. 1, may be contained in medical environment 200. As shown in FIG. 2B, medical environment 200 may be a patient room with a patient 205, one or more audio sensors 210, 215 (e.g., microphones), a sink 220, a soap dispenser 225, a door 240, and sanitation monitoring device 110. Medical environment 200 may include one or more sanitations devices, e.g., sink 220, soap dispenser 225, etc. A user may enter and exit the medical environment through the door 240. A user 230 may have a user device 130 on or near their person. In some embodiments, a medical environment may be an emergency room, operating room, or a patient room of a hospital or other medical facility.

In one or more embodiments, each of the sounds produced by the operation of the sanitation devices may be sensed by the sanitation monitoring device 110 and/or audio sensors 210, 215. For example, the sink 220 running or the soap dispenser 225 dispensing soap may produce a sound that is sensed by the microphones 210, 215, the sanitation monitoring device, and/or the user device 232.

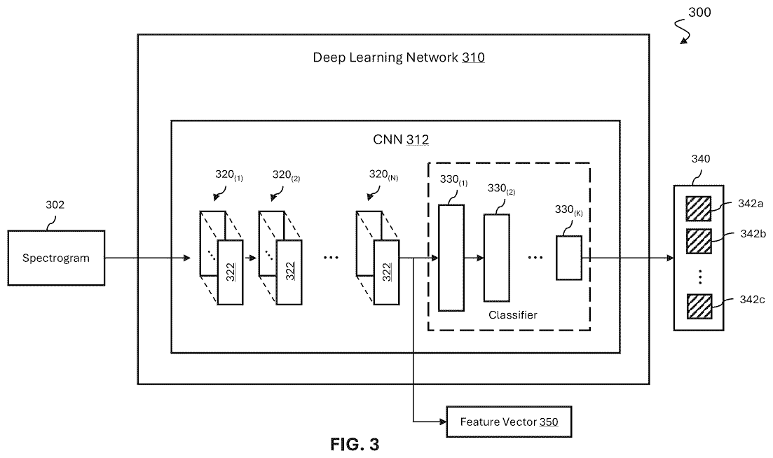

In one or more embodiments, the raw audio file from the audio sensors 210, 215 or the sensor 114 in the sanitation monitoring device may be further processed into a spectrogram depicting the frequency content of the recorded sound over time. In some embodiments, the sanitation monitoring system may continuously receive audio signals from the medical environment. A machine learning model, such as described in FIG. 3 below, may be used to segment or otherwise identify time frames of relevant sanitation activity in the medical environment. For example, a machine learning model may identify the time window during which a sink or soap dispenser was used. Alternatively, the sanitation monitoring system may start and stop listening based on the entry and exit of a person into a medical environment. For example, when a nurse enters patient room 200 recording may begin, and when the nurse leaves the patient room recording may cease. Then the machine learning model may determine when, if ever, the nurse engaged in sanitation activities, e.g., hand-washing.

As shown in FIG. 2B, in one or more embodiments, the sanitation device 225 may be in communication with the user device 130 via the network 190. In one or more embodiments, the user device may perform any one or more functions of the sanitation monitoring device described above, including tracking when the user uses a sanitation device when visiting a patient room. In one or more embodiments, each user associated with a respective user device may have an account accessible via the user device. In one or more embodiments, the user is a staff member of the medical facility. The staff member may enter and exit a plurality of patient rooms in the medical facility. Each time the staff member enters a patient room, the user device associated with the staff member may connect to a sanitation device in the patient room. When the staff member uses the sanitation device, the sanitation device may communicate such a sanitation event to the user device. As described above with respect to the sanitation monitoring event, the sanitation device may communicate to the user device whether the staff member uses the sanitation device when the staff member enters the patient room and whether the staff member uses the sanitation device when the staff member exits the patient room. This information can be collected for each patient room that the staff member visits. As shown in FIG. 2B, the compliance of the staff members with respect to using sanitation devices when entering patient rooms, when exiting patient rooms, and overall compliance with using sanitation devices may be displayed as a sanitation compliance score on the user device. This allows the staff member to view their sanitation performance and compliance with the sanitation requirements.

In one or more embodiments, the sanitation monitoring device may also use the visitor data, the sanitation event data, or the compliance scores to calculate a probability, or determine a likelihood, that the patient in the patient room will contract or experience a healthcare related infection. In one or more embodiments, the sanitation monitoring device may use the computer, which may be stored locally or remotely relative to the sanitation monitoring device, to process the collected data, calculate the compliance scores, and calculate, using the collected data or compliance scores, the probability that the patient in the patient room will contract a healthcare related infection. In one or more embodiments, the sanitation monitoring device and the computer may use a machine learning model to process the data and scores and determine the probability that the patient in the patient room will contract a healthcare related infection. The machine learning model may be trained with historical data regarding the relationship between the frequency with which visitors to a patient room use a sanitation device and the type and frequency with which the patient in the patient room contracts a healthcare related infection.

In one or more embodiments, the healthcare related infection may include central line-associated bloodstream infections (CLABSI), catheter-associated urinary tract infections (CAUTI), surgical site infections (SSI), ventilator-associated pneumonia (VAP), Clostridium difficile infections (CDI), methicillin-resistant Staphylococcus aureus (MRSA), and sepsis.

In one or more embodiments, the calculated probability that the patient in the patient room will experience a healthcare related infection may be displayed on the sanitation monitoring device, the user device, the computer, or may be transmitted to another device within the medical facility to alert staff members so that preventive or proactive measures and treatment may be taken or implemented to combat the healthcare related infection and keep the patient healthy.

FIG. 3 illustrates a neural network-based model configuration 300 for audio classification, according to one or more embodiments. The configuration 300 can be implemented by a deep learning network. The configuration 300 includes a deep learning network 310, which may include one or more CNNs 312. The CNN 312 is one example of a type of predictive model and/or machine learning model 170, which may be used in sanitation monitoring system 100. For simplicity of illustration and discussion, FIG. 3 illustrates one CNN 312. However, any suitable number of CNNs 312 (e.g., about 2, 3 or more) may be included. The configuration 300 can be trained for identification of various sounds (water running, soap dispensing, door opening/closing, etc.) and/or other features (sanitation-related or not) within a medical environment. The configuration 300 can be further trained for predicting a probability of infection risk for one or more patients in a medical environment such as a hospital. In one or more embodiments, the neural network-based model may be stored on computer 140 as machine learning model 170 and/or locally on the sanitation monitoring device 110, as described in FIG. 1.

The CNN 312 may include a set of N convolutional layers 320 followed by a set of K fully connected layers 330, where N and K may be any positive integers. The convolutional layers 320 are shown as 320(1) to 320(N). The fully connected layers 330 are shown as 330(1) to 330(K). Each convolutional layer 320 may include a set of filters 322 configured to extract features from an input 302 (e.g., spectrograms processed from an audio sample). The values N and K and the size of the filters 322 may vary depending on the use of the CNN. In some instances, the convolutional layers 320(1) to 320(N) and the fully connected layers 330(1) to 330(K−1) may be interspersed with rectified non-linear (ReLU) or leaky ReLU or other activation functions and/or batch normalization layers. The fully connected layers 330 may gradually shrink the high-dimensional output to a lower dimension or the dimension of the predicted result 340 (e.g., number of classes for a classification output). The fully connected layers 330 may also be referred to as a classifier. In some aspects, the fully convolutional layers 320 may additionally be referred to as representations or encodings or features, where the output of the fullu convolutional layers may be called a feature vector 350 or map.

When the prediction result 340 takes the form of classification output, it may indicate a confidence score (e.g., a probability) for each class 342 based on the input image 302. The classes 342 are shown as 342a, 342b, . . . , 342c. For example, when the CNN 312 is trained for classification of sounds in a medical environment, the classes 342 may indicate a first sound class 342a, a second sound class 342b, a third sound class 342c, a fourth found class 342d, or any other suitable class. A class 342 indicating a high confidence score indicates that the input spectrogram 302 or a section of the spectrogram 302 is likely to include a sound of the class 342. Conversely, a class 342 indicating a low confidence score indicates that the input image 302 or a section of the spectrogram 302 is unlikely to include a sound of the class 342. For example, if the spectrogram include the audio content for a sink running, then configuration 300 may generate a high probability for a sanitation device class of sounds, or high probability for a water running class. In this way, classes may be learned in a coarse-grained way (e.g., a class for sanitation device sounds) or more fine-grained way (e.g., a class for a water running, soap dispensing, etc.).

The CNN 312 can also output a feature vector 350 at the output of the last convolutional layer 320(N), though any of the layers in the CNN are feature vectors. A feature vector 350 or encodings or representations may encode some representation of sounds detected from the input spectrogram 302 or other data. For example, the feature vector 350 may encode representations of the sanitation device sounds or other sounds in the medical environment identified from the spectrogram 302, which may many sounds, such as a patient speaking while a sink is running. The feature vector 350 may encode some representation of the frequency content of the spectrogram associated with the source of sounds reflected in the spectrogram 302, as described herein. These representations can be decoded using a reversed CNN where the fully connected layers expand the low-dimensional representation to a higher dimension and transposed convolutional layers can expand feature layers up to the size of the original input, where pixel-wise outputs (e.g., binary segmentation map, multi-class segmentation map) can be generated.

The deep learning network 310 may implement or include any suitable type of learning network. For example, in some aspects, and as described in relation to FIG. 3, the deep learning network 310 could include a convolutional neural network 312. In addition, the deep learning network 310 may additionally or alternatively be or include a multi-class classification network, an encoder-decoder type network, a fully connected deep learning network, or any suitable network or means of identifying features within an image.

In some aspects, when the deep learning network 310 includes a fully-connected neural network, the fully connected neural network may transform the data not related to sounds in the medical environment or it may transform data derived from a spectrogram (e.g., the use of one or more sanitation devices) generated by another network, program, or human annotator. Data for detected objects may be concatenated into a layer (e.g., as an additional channel). For simple numeric features like time, patient characteristics, and other data-these can be concatenated into fully connected layers. For example, the fully connected neural network may transform information about a patient, such as age, weight, medical status, or other low-dimensional data.

In some aspects, when the deep learning network 310 includes an encoder-decoder network, the network may include two components. One component may be a constricting component or encoder, in which a large image, such as the spectrogram 302, may be convolved by several convolutional layers 320 such that the size of the spectrogram 302 changes in relation to the depth of the network layer. For instance, the CNN 312 may be the encoder. The spectrogram 302 may then be represented in a low dimensional space, or a flattened space. From this flattened space, an additional component or decoder may expand the flattened space to the original size of the spectrogram 302. For instance, the reverse of the CNN 312 may be the decoder. In some aspects, the encoder-decoder network may reconstruct the input spectrogram 302. In some aspects, the encoder-decoder network may segment the spectrogram 302 into patches. In some aspects of the present disclosure, the deep learning network 310 may include a multi-class classification network. In that aspect, the multi-class classification network may include an encoder path. For example, the spectrogram 302 may be a high dimensional, dense spectrogram. The spectrogram 302 may then be processed with the convolutional layers 320 such that the size is reduced. The resulting low dimensional representation of the spectrogram 302 may be used to generate the feature vector 350 shown in FIG. 3. The low dimensional representation of the spectrogram 302 may additionally be used by the fully connected layers 330 to regress and output one or more classes 342. In some regards, the fully connected layers 330 may process the output of the convolutional layers 320. The fully connected layers 330 may additionally be referred to as task layers or regression layers, among other terms.

Any suitable combination or variations of the deep learning network 310 described is fully contemplated. For example, the deep learning network may include fully convolutional networks or layers or fully connected networks or layers or a combination of the two. In addition, the deep learning network may include a multi-class classification network, an encoder-decoder network, or any combination of networks. The process of training the deep learning network 310 includes adjusting its parameters, or more particularly the weights and biases, which control the operation of activation functions in the neural network. In supervised learning, the training process automatically adjusts the weights and the biases, such that when presented with the input data, the neural network accurately provides the corresponding expected output data. In order to do this, the value of the loss functions, or errors, are computed based on a difference between predicted output data and the expected output data. The value of the loss function may be computed using functions such as the negative log-likelihood loss, the mean squared error, or the Huber loss, or the cross-entropy loss. During training, the value of the loss function is typically minimized. Various methods are known for solving the loss minimization problem such as gradient descent, Quasi-Newton methods, and so forth. Various algorithms have been developed to implement these methods and their variants including but not limited to Stochastic Gradient Descent “SGD”, batch gradient descent, mini-batch gradient descent, Gauss-Newton, Levenberg Marquardt, Momentum, Adam, Nadam, Adagrad, Adadelta, RMSProp, and Adamax “optimizers” These algorithms compute the derivative of the loss function with respect to the model parameters using the chain rule. This process is called backpropagation since derivatives are computed starting at the last layer or output layer, moving toward the first layer or input layer. These derivatives inform the algorithm how the model parameters must be adjusted in order to minimize the error function. The training process is performed iteratively by making adjustments to the weights and biases in each iteration. Training is terminated when the error, or difference between the predicted output data and the expected output data, is within an acceptable range for the training data, or for some validation data. Subsequently the neural network may be deployed, and the trained neural network makes predictions on new input data using the trained values of its parameters. If the training process was successful, the trained neural network accurately predicts the expected output data from the new input data.

In some embodiments, machine learning models may be used to generate an infection risk or other score from tracked sanitation events (e.g., sanitation events identified using the deep learning network 310 discussed above). An infection risk may be associated with an individual patient, a group of patients (e.g., those on the same floor, wing, or other infrastructure division or those having a similar disease profile or that are similarly immunocompromised), or an entire body of patients within a single facility, such as a hospital. In some embodiments, an infection risk may be determined by one or more machine learning models, such as regression models (e.g., linear, multi-linear, polynomial, logistic, gradient boosting etc.) decision trees, support vector machines, random forest, and the like.

Input to a machine learning model for predicting infection risk may take many forms, including patient electronic medical records, sanitation compliance score(s), number of patient interactions, etc. For example, input may include aggregate data of sanitation events, as described herein, and/or resulting compliance scores. In other examples, individual or aggregate patient information may also be used. For example, some medical environments may treat patients with a greater susceptibility to infections than others. For example, average patient age may serve as input to a machine learning model, recognizing, for example, that older patients tend to be more at risk for infection than younger patients. Similarly, a numerical factor representative of the degree of immunocompromise in a patient population may serve as an input to a machine learning model, recognizing, for example, that patient groups with a greater degree compromised immune systems tend to be at greater risk of infection than patient groups with less compromised immune systems. In some embodiment, one or more data from patient electronic medical records (EMR) may be used as input to the machine learning model.

In some embodiments, machine learning model(s) may output one or more scores indicative of an infection risk. For example, the output may be a number between 0 and 1, where 1 indicated a 100% likelihood of infection, and 0% represents a 0% likelihood of infection. Additional scores may also be output by the machine learning model. For example, an additional score may indicate the severity of an infections. Thus, in some instances, a machine learning model may predict a lower/higher risk of infection and a lesser/greater severity infection. It should be appreciated that other numerical ranges and additional scores are contemplated by the present disclosure.

In some embodiments, the output of the deep learning network 310 described in FIG. 3 may be used as input into a machine learning model for generating infection risk. For example, the deep learning network 310 may be used to determine a number of sanitation events which may be combined with user visit data to determine a compliance score. The compliance score may be provided as in put to the machine learning model to output an infection risk score.

In some embodiments, the machine learning model(s) may be produced or trained using known techniques with historical data and infections occurrences. For example, a threshold in a step of a decision tree may be determined by updating the threshold value until the decision tree accurately predicts the historical infection rate from ground truth data. As another example, the parameters of a regression model may be updated until the model accurately predicts the historical infection rate from ground truth data from the historical data. Historical data may include user visit (entrance and exit) data to patient rooms or other medical environments, as well as other patient information and clinical data.

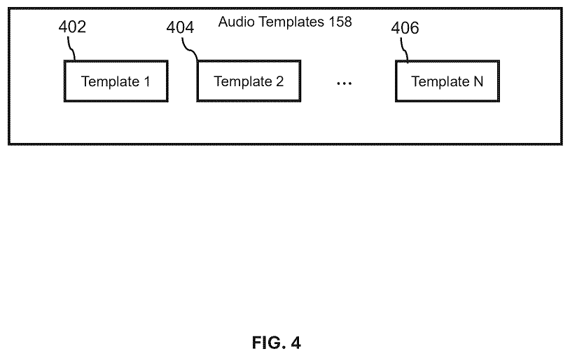

FIG. 4 illustrates exemplary audio templates 158, according to one or more embodiments. In one or more embodiments, audio templates 158 may be used to determine the occurrence of a sanitation event in a medical environment. One or more audio templates may be pre-programmed and/or recorded for use by computer 140 or sanitation monitoring device 110 to make a comparison. In some embodiments, audio templates 158 include a first template 402, second template 404, and N-th template 406. For example, first template 402 may be a raw audio file or spectrogram of water running from a sink, second template 404 may be a raw audio file or spectrogram of soap dispensing from a soap dispenser, and N-th template 406 may be a raw audio file or spectrogram of sounds produced by any other sanitation device.

In some embodiments, once a sound has been recorded in the medical environment, its features may be compared to each template to determine which of the associated sanitation devices, if any, was used. In addition, the number of time a sanitation device is used may be counted. For example, a nurse washing his/her hands upon entering and exiting a patient room would yield a count of two for the number of hand washing events. However, soap dispensing may only be detected once, which would imply one of the hand washings occurred without the use of soap. This fact may be used to alter or modify a sanitation compliance score or to flag to a user the failure to use soap.

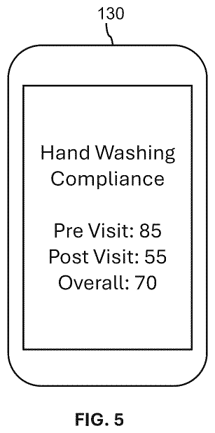

FIG. 5 illustrates a display of sanitation compliance scores generated by the sanitation monitoring system of FIG. 1, according to one or more embodiments. Compliance scores may be beneficially provided to users on their user device 130. A user interface on the user device 130 may give daily, weekly, monthly sanitation compliance scores. In some embodiments, alerts may be provided to users through their user device indicating a failure to follow sanitation procedures. For example, if a doctor were to enter or leave a patient's room without using soap during handwashing an alert may be sent in real time to the doctor notifying them of the mistake.

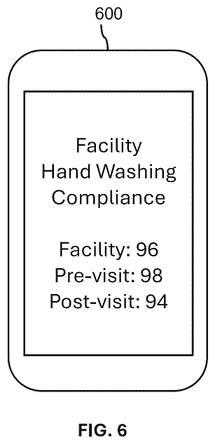

FIG. 6 illustrates a display of sanitation compliance scores generated by the sanitation monitoring system of FIG. 1 for a supervisor, according to one or more embodiments. Scores and other information described herein may be displayed on the user interface of a user device 600. Compliance scores may be beneficially provided to supervisors or other managerial personnel, presenting aggregate or individualized compliance data. For example, facility-wide, e.g., hospital-wide, data may be viewed. Reports may be displayed listing medical personnel with the lowest compliance scores.

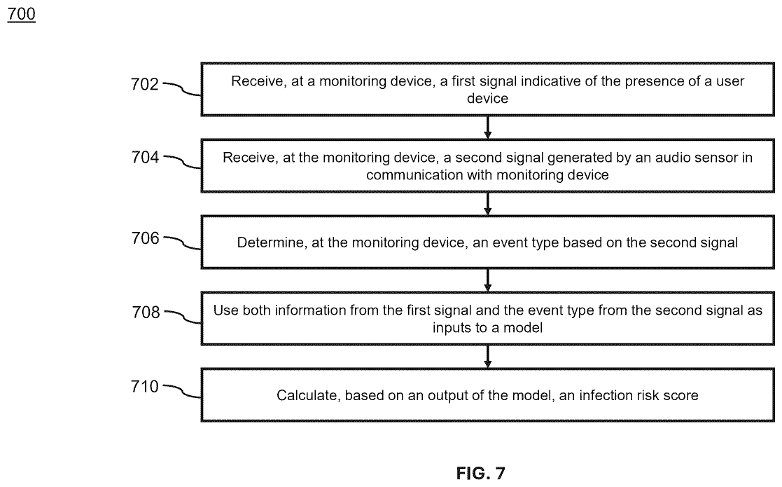

FIG. 7 illustrates a method 700 of monitoring sanitation events in a medical environment, according to one or more embodiments. In some embodiments, the medical environment may be an emergency room, an operating room, or a patient room of a hospital. One or more of the processes of method 700 may be implemented, at least in part, in the form of executable code stored on non-transitory, tangible, machine-readable media that when run by one or more processors may cause the one or more processors to perform one or more of the processes. In some embodiments, method 700 corresponds to the operation of one or more components of the sanitation monitoring system 100 that performs sanitation monitoring.

In some embodiments, method 700 is performed by a system and/or device such as a sanitation monitoring device 110 or another device or combination of devices. Inputs (e.g., audio received at sensor 114 and/or audio sensors 210, 215) may be received via a data interface integrated with a device.

As illustrated, the method 700 includes a number of enumerated steps, but aspects of the method 700 may include additional steps before, after, and in between the enumerated steps. In some aspects, one or more of the enumerated steps may be omitted or performed in a different order.

At step 702, a monitoring device (e.g., sanitation monitoring device 110 in FIGS. 1-2) receives a first signal indicative of the presence of a user device (e.g., 130 in FIGS. 1-2). For example, the first signal may be a short-range wireless signal, e.g., a Bluetooth signal, from user device 130. In some embodiments, information about the user may be associated with the user device. For example, whether the user is a doctor, nurse, or non-medical personnel, such as a visitor. In some embodiments, if a user with a low compliance score is detected entering a patient room, then an alert may be sent to their user device to remind them to wash their hands after entry and before exiting the patient room.

At step 704, the monitoring device (e.g., sanitation monitoring device 110 in FIGS. 1-2) receives a second signal (e.g., raw audio or a spectrogram) generated by an audio sensor (e.g., audio sensors 210, 215 and/or sensor 114) in communication with the monitoring device. The second signal may be indicative of a sanitation action at a sanitation device (e.g., a soap dispenser, hand sanitizer dispenser, or handwashing sink) by a user within audio range of the monitoring device. In some embodiments, sanitation monitoring device may process a raw audio file into a spectrogram depicting the frequency content of the audio signal overtime.

At step 706, the monitoring device (e.g., sanitation monitoring device 110 in FIGS. 1-2) determines an event type (e.g., sanitation event such as soap dispending or sink running) based on the second signal. In some embodiments, a neural network-based model (e.g., 300 as described in FIG. 3; also called a machine learning model 170) may be used to classify a spectrogram to produce an event type. In some aspects, the sanitation monitoring device may further segment the second signal using a machine learning model and classifying each segment of the audio file or spectrogram. For example, two hand washing events may be present in an audio file. The machine learning model may identify the time windows that include each hand washing event. In some aspects, a comparison of the features in the second signal, or the spectrogram generated therefrom, may be compared to audio templates 158 to determine a level of similarity and thereby a predict of the event type.

At step 708, a device (e.g., sanitation monitoring device 110 or computer 140 in networked system 100) uses both information from the first signal and the event type from the second signal as inputs to a model. For example, the model may be a machine learning model as described herein, e.g., a regression model, decision tree, random forest etc. The model may receive information including a count of sanitation events along with the number of users visiting a medical environment, such as a patient room. The number of users may be determined from the first signal through a Bluetooth signal detected when a visitor enters a medical environment. In some embodiments, counts of sanitation events may be associated with each individual user such that a count of sanitation events for a particular user is available (e.g., counting sanitation event for each nurse or doctor). For example, using the counters maintained by the sanitation monitoring device, the device may compute compliance scores for a user or for all users within a wider medical environment, such as a hospital.

At step 710, a device (e.g., sanitation monitoring device 110 or computer 140 in networked system 100) calculates, based on an output of the model (e.g., machine learning model 170), an infection risk score. In some embodiments, an infection risk score indicates a likelihood of a healthcare infection within a medical environment. In some embodiments, the output of the model may be the infection risk score. For example, the output of the model may be a number between 0 and 1, indicating the probability of infection. In some embodiments, the output of the model may be used in the evaluation of a decision tree to determine an infection risk score. For example, the output of the model may be compared to a threshold evaluated at a step in a decision tree.

In some embodiments, the sanitation monitoring device may generate a healthcare infection risk, e.g., using machine learning model 170. For example, using patient health information (e.g., contained in electronic medical records) and the compliance scores of medical professionals attending a patient, a machine learning model may generate a probability of infection for the patient. In some embodiments, machine-learning model that is trained by processing historical data that correlates a number of visitors and sanitation events with healthcare infections. In this way, the machine learning model can make predictions of future infections based on current data.

In some embodiments, after determining the infection risk score an alert may be sent to a user, e.g., through a user interface similar to the ones described in FIGS. 5-6, either reminding the user to sanitize their hands or to notify them of an increased infection risk. In some embodiments, the infection risk is calculated iteratively in real-time as new data is received over the course of minutes, days, weeks, etc. When the infection risk increases beyond a certain threshold or the rate of change exceeds a threshold, then a notification is sent to a user to reallocate sanitation resources or to identify a higher infection risk.

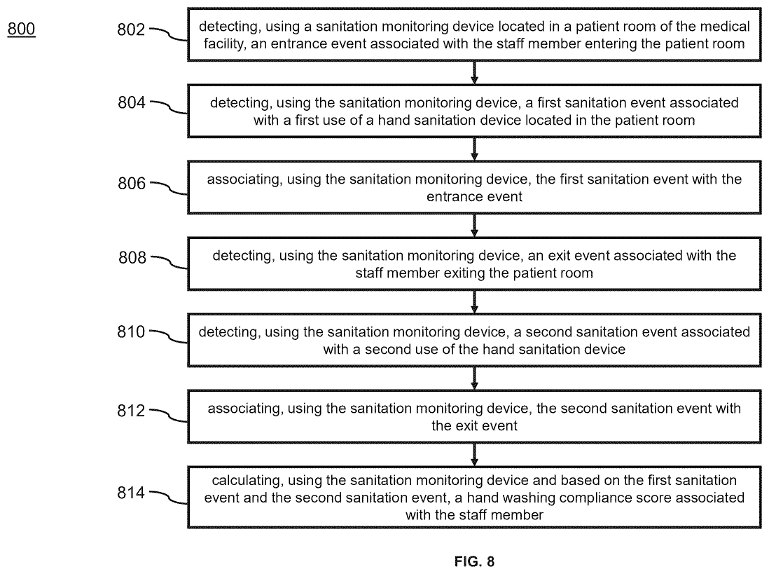

FIG. 8 illustrates a method of tracking hand sanitation of staff members in a medical facility using the sanitation monitoring system, according to one or more embodiments. In one or more embodiments, the method of FIG. 8 includes: detecting, using a sanitation monitoring device located in a patient room of the medical facility, an entrance event associated with the staff member entering the patient room; detecting, using the sanitation monitoring device, a first sanitation event associated with a first use of a hand sanitation device located in the patient room; associating, using the sanitation monitoring device, the first sanitation event with the entrance event; detecting, using the sanitation monitoring device, an exit event associated with the staff member exiting the patient room; detecting, using the sanitation monitoring device, a second sanitation event associated with a second use of the hand sanitation device; associating, using the sanitation monitoring device, the second sanitation event with the exit event; and calculating, using the sanitation monitoring device and based on the first sanitation event and the second sanitation event, a hand washing compliance score associated with the staff member.

In one or more embodiments, detecting the entrance event comprises detecting, using the sanitation monitoring device, that a user device associated with the staff member has entered the patient room. In one or more embodiments, detecting that the user device has entered the patient room comprises establishing, using the sanitation monitoring device, a short-range wireless connection between the sanitation monitoring device and the user device. In one or more embodiments, detecting the exit event comprises detecting, using the sanitation monitoring device, that the user device associated with the staff member has exited the patient room. In one or more embodiments, detecting that the user device has exited the patient room comprises determining, using the sanitation monitoring device, that the short-range wireless connection between the sanitation monitoring device and the user device has terminated. In one or more embodiments, the sanitation monitoring device comprises a sensor, and detecting the first sanitation event and the second sanitation event comprises detecting, using the sensor, a sound associated with the first and second use of the hand sanitation device, respectively. In one or more embodiments, displaying, using the sanitation monitoring device, the hand washing compliance score associated with the staff member. In one or more embodiments, displaying, using the user device, the hand washing compliance score associated with the staff member.

In some examples, the sanitation monitoring system does not associate specific persons with specific hand washing events. Rather, the system tracks aggregate visitors and aggregate hand sanitizing events.

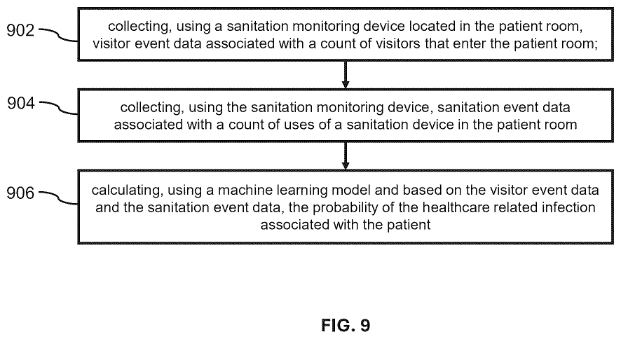

FIG. 9 illustrates a method of calculating a probability of a healthcare related infection occurring with respect to a patient of the medical facility using the sanitation monitoring system, according to one or more embodiments. In one or more embodiments, the method of FIG. 9 includes: collecting, using a sanitation monitoring device located in the patient room, visitor event data associated with a count of visitors that enter the patient room; collecting, using the sanitation monitoring device, sanitation event data associated with a count of uses of a sanitation device in the patient room; and calculating, using a model and based on the visitor event data and the sanitation event data, the probability of the healthcare related infection associated with the patient. The model may be derived using machine learning techniques. The model may also be updated with new data that correlates hand sanitation usage, visitors, and healthcare infections.

In one or more embodiments, collecting visitor event data comprises: detecting, using the sanitation monitoring device and via short-range wireless connection, user devices associated with the visitors that enter the patient room; and incrementing, based on each detection of the user devices associated with the visitors that enter the patient room, the count of visitors that enter the patient room. Other techniques for detecting visitors are contemplated. In one or more embodiments, visitors include medical staff of the medical facility and guests of the medical facility.

In one or more embodiments, collecting sanitation event data comprises: detecting, using a sensor of the sanitation monitoring device, a sound associated with use of the sanitation device; and incrementing, based on each detection of use of the sanitation device, the count of uses of the sanitation device in the patient room. Collecting sanitation event data may also be done using a detector installed within a sanitation device that transmits a signal to the sanitation monitoring device when the sanitation device is used.

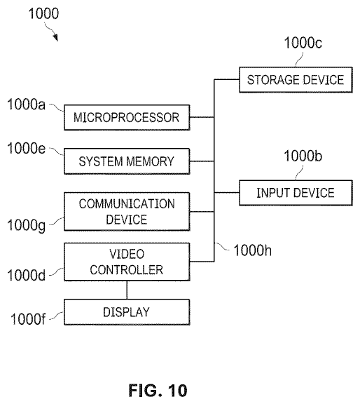

FIG. 10 illustrates a node for implementing one or more embodiments of the present disclosure. The node 1000 includes a microprocessor 1000a, an input device 1000b, a storage device 1000c, a video controller 1000d, a system memory 1000e, a display 1000 f, and a communication device 1000g all interconnected by one or more buses 1000h. In several example embodiments, the storage device 1000c may include a hard drive, CD-ROM, optical drive, any other form of storage device and/or any combination thereof. In several example embodiments, the storage device 1000c may include, and/or be capable of receiving, CD-ROM, DVD-ROM, or any other form of computer-readable medium that may contain executable instructions. In several example embodiments, the communication device 1000g may include a modem, network card, or any other device to enable the node to communicate with other nodes. In several example embodiments, any node represents a plurality of interconnected (whether by intranet or Internet) computer systems, including without limitation, personal computers, mainframes, PDAs, smartphones, and cell phones.

In several example embodiments, one or more of the components of the systems described above, and/or any combination thereof, include at least the node 1000 and/or components thereof, and/or one or more nodes that are substantially similar to the node 1000 and/or components thereof. In several example embodiments, one or more of the above-described components of the node 1000 and/or the system include respective pluralities of same components.

In several example embodiments, one or more of the applications, systems, and application programs described above, and/or any combination thereof, include a computer program that includes a plurality of instructions, data, and/or any combination thereof; an application written in, for example, Arena, Hypertext Markup Language (HTML), Cascading Style Sheets (CSS), JavaScript, Extensible Markup Language (XML), asynchronous JavaScript and XML (Ajax), and/or any combination thereof; a web-based application written in, for example, Java or Adobe Flex, which in several example embodiments pulls real-time information from one or more servers, automatically refreshing with latest information at a predetermined time increment; or any combination thereof.

In several example embodiments, a computer system typically includes at least hardware capable of executing machine readable instructions, as well as the software for executing acts (typically machine-readable instructions) that produce a desired result. In several example embodiments, a computer system may include hybrids of hardware and software, as well as computer sub-systems.

In several example embodiments, hardware generally includes at least processor-capable platforms, such as client-machines (also known as personal computers or servers), and hand-held processing devices (such as smart phones, tablet computers, personal digital assistants (PDAs), or personal computing devices (PCDs), for example). In several example embodiments, hardware may include any physical device that can store machine-readable instructions, such as memory or other data storage devices. In several example embodiments, other forms of hardware include hardware sub-systems, including transfer devices such as modems, modem cards, ports, and port cards, for example.

In several example embodiments, software includes any machine code stored in any memory medium, such as RAM or ROM, and machine code stored on other devices (such as flash memory, or a CD ROM, for example). In several example embodiments, software may include source or object code. In several example embodiments, software encompasses any set of instructions capable of being executed on a node such as, for example, on a client machine or server.

In several example embodiments, combinations of software and hardware could also be used for providing enhanced functionality and performance for certain embodiments of the present disclosure. In an example embodiment, software functions may be directly manufactured into a silicon chip. Accordingly, it should be understood that combinations of hardware and software are also included within the definition of a computer system and are thus envisioned by the present disclosure as possible equivalent structures and equivalent methods.

In several example embodiments, computer readable mediums include, for example, passive data storage, such as a random-access memory (RAM) as well as semi-permanent data storage such as a compact disk read only memory (CD-ROM). One or more example embodiments of the present disclosure may be embodied in the RAM of a computer to transform a standard computer into a new specific computing machine. In several example embodiments, data structures are defined organizations of data that may enable an embodiment of the present disclosure. In an example embodiment, a data structure may provide an organization of data, or an organization of executable code.

In several example embodiments, any networks and/or one or more portions thereof may be designed to work on any specific architecture. In an example embodiment, one or more portions of any networks may be executed on a single computer, local area networks, client-server networks, wide area networks, internets, hand-held and other portable and wireless devices, and networks.

In several example embodiments, a database may be any standard or proprietary database software. In several example embodiments, the database may have fields, records, data, and other database elements that may be associated through database specific software. In several example embodiments, data may be mapped. In several example embodiments, mapping is the process of associating one data entry with another data entry. In an example embodiment, the data contained in the location of a character file can be mapped to a field in a second table. In several example embodiments, the physical location of the database is not limiting, and the database may be distributed. In an example embodiment, the database may exist remotely from the server, and run on a separate platform. In an example embodiment, the database may be accessible across the Internet. In several example embodiments, more than one database may be implemented.

In several example embodiments, a plurality of instructions stored on a computer readable medium may be executed by one or more processors to cause the one or more processors to carry out or implement in whole or in part the above-described operation of each of the above-described example embodiments of the system, the method, and/or any combination thereof. In several example embodiments, such a processor may include one or more of the microprocessor 1000a, any processor(s) that are part of the components of the system, and/or any combination thereof, and such a computer readable medium may be distributed among one or more components of the system. In several example embodiments, such a processor may execute the plurality of instructions in connection with a virtual computer system. In several example embodiments, such a plurality of instructions may communicate directly with the one or more processors, and/or may interact with one or more operating systems, middleware, firmware, other applications, and/or any combination thereof, to cause the one or more processors to execute the instructions.

The present disclosure also introduces an apparatus, which apparatus has been described according to one or more aspects of the present disclosure.

The present disclosure also introduces a system, which system has been described according to one or more aspects of the present disclosure.

The present disclosure also introduces a method, which method has been described according to one or more aspects of the present disclosure.

The present disclosure also introduces an assembly, which assembly has been described according to one or more aspects of the present disclosure.

It is further understood that variations may be made in the foregoing without departing from the scope of the disclosure.

In one or more embodiments, the elements and teachings of the various embodiments disclosed herein may be combined in whole or in part in some or all of said embodiment(s). In addition, one or more of the elements and teachings of the various embodiments disclosed herein may be omitted, at least in part, or combined, at least in part, with one or more of the other elements and teachings of said embodiment(s).

Any spatial references such as, for example, “upper,” “lower,” “above,” “below,” “between,” “bottom,” “vertical,” “horizontal,” “angular,” “upwards,” “downwards,” “side-to-side,” “left-to-right,” “left,” “right,” “right-to-left,” “top-to-bottom,” “bottom-to-top,” “top,” “bottom,” “bottom-up,” “top-down,” etc., are for the purpose of illustration only and do not limit the specific orientation or location of the structure described above.

In one or more embodiments, while different steps, processes, and procedures are described as appearing as distinct acts, one or more of the steps, one or more of the processes, or one or more of the procedures may also be performed in different orders, simultaneously or sequentially. In one or more embodiments, the steps, processes, or procedures may be merged into one or more steps, processes, or procedures. In one or more embodiments, one or more of the operational steps in each embodiment may be omitted. Moreover, in some instances, some features of the present disclosure may be employed without a corresponding use of the other features. Moreover, one or more of the embodiments disclosed above, or variations thereof, may be combined in whole or in part with any one or more of the other embodiments described above, or variations thereof.

Although various embodiments have been disclosed in detail above, the embodiments disclosed are exemplary only and are not limiting, and those skilled in the art will readily appreciate that many other modifications, changes, and substitutions are possible in the embodiments without materially departing from the novel teachings and advantages of the present disclosure. Accordingly, all such modifications, changes, and substitutions are intended to be included within the scope of this disclosure as defined in the following claims. In the claims, means-plus-function clauses are intended to cover the structures described herein as performing the recited function and not only structural equivalents, but also equivalent structures. Moreover, it is the express intention of the applicant not to invoke 35 U.S.C. § 112(f) for any limitations of any of the claims herein, except for those in which the claim expressly uses the word “means” together with an associated function.

What is claimed is:

1. A method for monitoring sanitation events in a medical environment, the method comprising:
receiving, at a monitoring device, a first signal indicative of the presence of a user device;
receiving, at the monitoring device, a second signal generated by an audio sensor in communication with monitoring device, wherein the second signal is indicative of a sanitation action by a user within audio range of the monitoring device;
determining, at the monitoring device, an event type based on the second signal;
using both information from the first signal and the event type from the second signal as inputs to a model; and
calculating, based on an output of the model, an infection risk score.

2. The method of claim 1, wherein the first signal is a Bluetooth signal from the user device.

3. The method of claim 1, wherein the model is a machine-learning model that is produced by processing historical data that correlates a number of visitors and sanitation events with healthcare infections.

4. The method of claim 1, wherein the medical environment is an emergency room, an operating room, or a patient room of a hospital.

5. The method of claim 1, wherein the event type is determined by:
generating, using a neural network-based model, one or more classifications based on the second signal, wherein the one or more classifications indicate the source of a sound in the medical environment.

6. The method of claim 5, wherein the source is a sanitation device.

7. The method of claim 6, wherein the sanitation device is one of: a hand sanitizer or a handwashing sink.

8. The method of claim 1, wherein the event type is determined by:
comparing the second signal with one or more audio templates.

9. The method of claim 1, wherein the infection risk score indicates a likelihood of a healthcare infection within the medical environment.

10. A system comprising:
a memory configured to store instructions for a model;
a processor coupled to the memory and configured to read the instructions from the memory to cause the system to perform operations, the operations comprising:
receive, at a monitoring device, a first signal indicative of the presence of a user device;
receive, at the monitoring device, a second signal generated by an audio sensor in communication with monitoring device, wherein the second signal is indicative of a sanitation action by a user within audio range of the monitoring device;
determine, at the monitoring device, an event type based on the second signal;
use both information from the first signal and the event type from the second signal as inputs to the model; and
calculate, based on an output of the model, an infection risk score.

11. The system of claim 10, wherein the first signal is a Bluetooth signal from the user device.

12. The system of claim 10, wherein the model is a machine-learning model that is produced by processing historical data that correlates a number of visitors and sanitation events with healthcare infections.

13. The system of claim 10, wherein the medical environment is an emergency room, an operating room, or a patient room of a hospital.

14. The system of claim 10, wherein the operations further comprising determining the event type by:
generating, using a neural network-based model, one or more classifications based on the second signal, wherein the one or more classifications indicate the source of a sound in the medical environment.

15. The system of claim 14, wherein the source is a sanitation device.

16. The system of claim 15, wherein the sanitation device is one of: a hand sanitizer or a handwashing sink.

17. The system of claim 10, wherein the event type is determined by operations further comprising:
comparing the second signal with one or more audio templates.

18. The system of claim 10, wherein the infection risk score indicates a likelihood of a healthcare infection within the medical environment.

19. A non-transitory machine-readable medium comprising a plurality of instructions, executable by one or more processors, wherein the plurality of instructions are configurable to cause the one or more processors to perform operations comprising:
receive, at a monitoring device, a first signal indicative of the presence of a user device;
receive, at the monitoring device, a second signal generated by an audio sensor in communication with monitoring device, wherein the second signal is indicative of a sanitation action by a user within audio range of the monitoring device;
determine, at the monitoring device, an event type based on the second signal;
use both information from the first signal and the event type from the second signal as inputs to a model; and
calculate, based on an output of the model, an infection risk score.

20. The non-transitory machine-readable medium of claim 19, wherein the first signal is a Bluetooth signal from the user device.
